(19) United States
(12) Patent Application Publication
(10) Pub. No.: US 2015/0255730 A1
(43) Pub. Date: Sep. 10, 2015

(54) BIPHENYL DERIVATIVE AND ORGANIC ELECTROLUMINESCENT DEVICE

(71) Applicant: Industrial Technology Research Institute, Hsinchu (TW)

(72) Inventors: Ya-Ting Chuang, Taipei City (TW); Jiun-Haw Lee, Taipei City (TW); Man-Kit Leung, Taipei City (TW); Tien-Lung Chiu, Taipei City (TW); Chi-Feng Lin, New Taipei City (TW); Jau-Jiun Huang, Kaohsiung City (TW)

(21) Appl. No.: 14/316,808

(22) Filed: Jun. 27, 2014

(30) Foreign Application Priority Data
Mar. 7, 2014 (TW) 103107957

(51) Int. Cl. H01L 51/00; H01L 51/52; C07D 249/08; H01L 51/50; C07D 235/20; C07D 271/107

(52) U.S. Cl. CPC H01L 51/0067; C07D 235/20; C07D 271/107; C07D 249/08; H01L 51/0072; H01L 51/007; H01L 51/5056; H01L 51/5092; H01L 51/5072; H01L 51/5215; H01L 51/5234; H01L 51/5028; H01L 2051/0063

(57) ABSTRACT

A biphenyl derivative is provided, which is shown in formula (1):

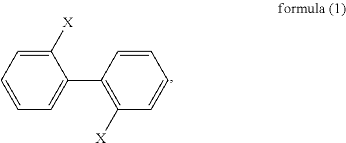

wherein X represents one of the groups shown in formula (2) to formula (5):

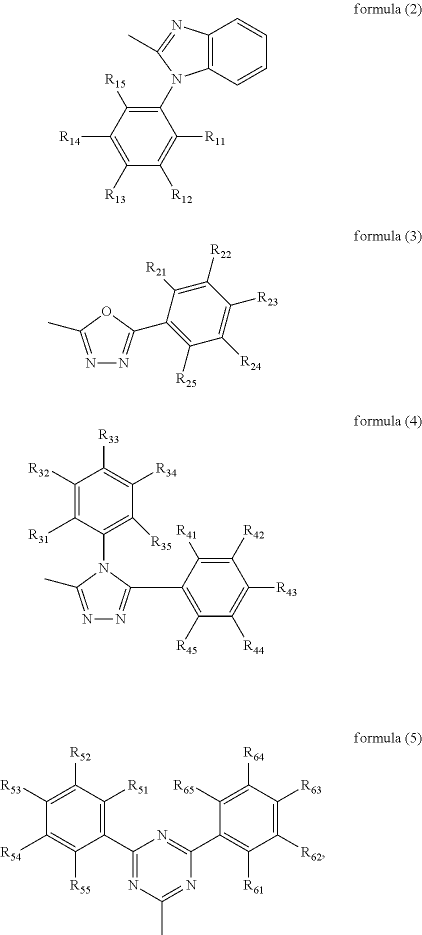

and

R₁₁ to R₁₅, R₂₁ to R₂₅, R₃₁ to R₃₅, R₄₁ to R₄₅, R₅₁ to R₅₅, and R₆₁ to R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group.

### CROSS-REFERENCE TO RELATED APPLICATION

This application claims the priority benefit of Taiwan application serial no. 103107957, filed on Mar. 7, 2014. The entirety of the above-mentioned patent application is hereby incorporated by reference herein and made a part of this specification.

### TECHNICAL FIELD

This disclosure relates to a biphenyl derivative and an organic electroluminescent device including the biphenyl derivative.

### BACKGROUND

An electroluminescent device is a semiconductor device capable of converting electrical energy into light with high conversion efficiency, which is commonly used as the luminous elements in indication lights, display panels, and optical reading/writing heads, etc. The electroluminescent device, having characteristics such as free viewing angle, simple fabrication process, low production cost, fast response, wide operation temperature range, and full color display, etc., is expected to become the mainstream of new flat-panel displays.

Generally speaking, an organic electroluminescent device includes an anode, an organic luminescent layer, and a cathode, wherein the organic luminescent layer includes a host material and a guest material. Typically the hole and electron in the organic electroluminescent device are transported to the host material to be combined for generating energy, which is transferred to the guest material for generating light. Therefore, the host material needs to have a favorable electron-hole transport property and a triplet energy level higher than or equal to that of the guest material, so as to prevent energy loss that results from energy transfer from dopant back to triplet state of host material (hereinafter referred to as reverse energy transfer).

Most of the current red and green phosphorescent light emitting diodes (LEDs) have good lifespan and performance. For blue phosphorescent LEDs, however, the guest material has higher triplet energy level than the guest materials of red and green LEDs. As a result, the aforementioned energy return phenomenon usually causes the blue phosphorescent LEDs to have low luminous efficiency (also called current efficiency) and short lifespan, etc. Thus, a host material that satisfies both the requirements of high triplet energy level and thermal stability is required.

### SUMMARY

The disclosure provides a biphenyl derivative.

The disclosure further provides an organic electroluminescent device having an organic luminescent material that includes the biphenyl derivative.

The biphenyl derivative of the disclosure is shown in formula (1):

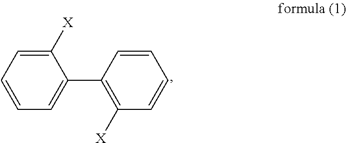

wherein X represents one of the groups shown in formula (2) to formula (5):

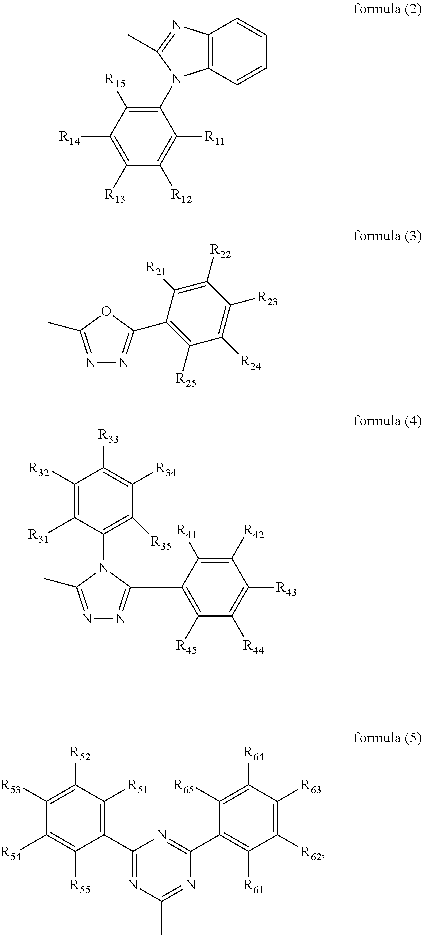

and R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group.

An organic electroluminescent device of the disclosure includes a first electrode layer, a second electrode layer, and an organic luminescent unit. The organic luminescent unit is disposed between the first electrode layer and the second electrode layer. The organic luminescent unit includes the biphenyl derivative shown in formula (1):

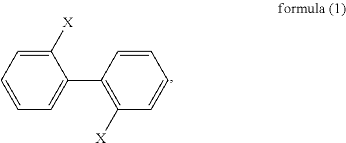

wherein X represents one of the groups shown in formula (2) to formula (5):

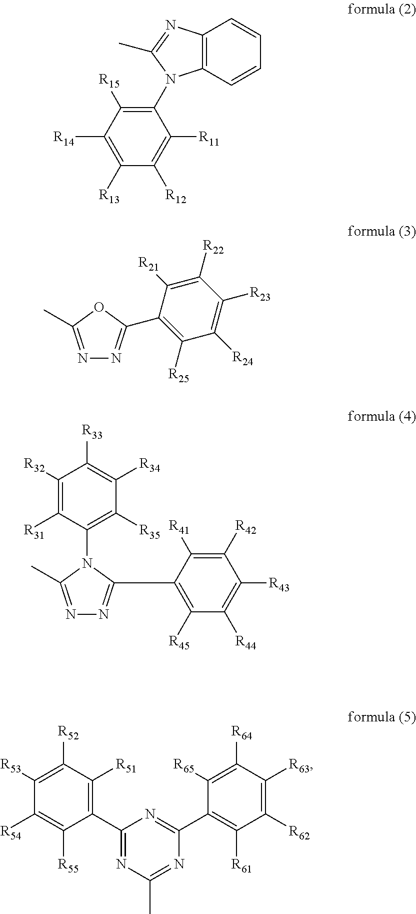

and R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group.

Another organic electroluminescent device of the disclosure includes a first electrode layer, a second electrode layer, and an organic luminescent unit. The organic luminescent unit is disposed between the first electrode layer and the second electrode layer. The organic luminescent unit includes an organic luminescent layer. The organic luminescent layer includes a host material and a guest material. The host material includes the biphenyl derivative shown in formula (1):

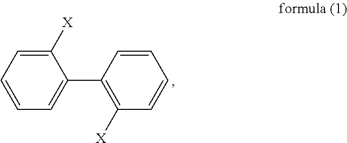

wherein X represents one of the groups shown in formula (2) to formula (5):

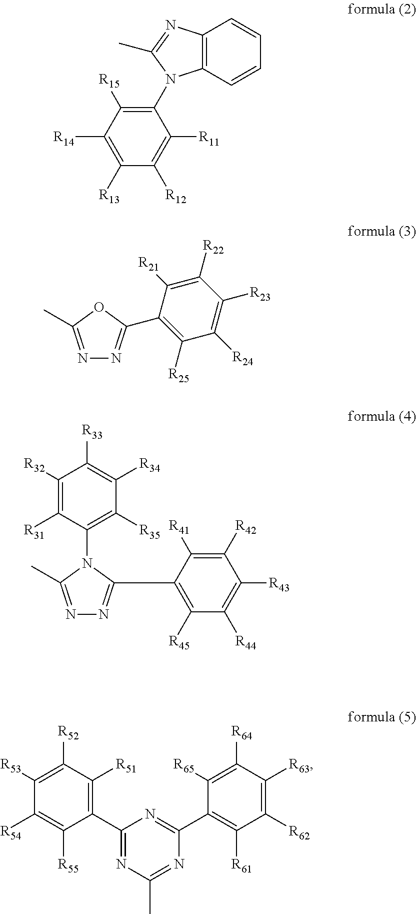

and R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group.

To make the disclosure more comprehensible, several embodiments accompanied with drawings are described in detail as follows.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a schematic top view of a molecular structure of a biphenyl derivative according to one embodiment of the disclosure.

FIG. 2 is a schematic side view of the molecular structure of the biphenyl derivative according to one embodiment of the disclosure.

FIG. 3 is a schematic cross-sectional view of an organic electroluminescent device according to one embodiment of the disclosure.

FIG. 4 is a schematic cross-sectional view of an organic electroluminescent device according to another embodiment of the disclosure.

FIG. 5 is a schematic cross-sectional view of an organic electroluminescent device according to yet another embodiment of the disclosure.

### DETAILED DESCRIPTION OF DISCLOSED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### [Organic Luminescent Material]

According to one embodiment of the disclosure, an organic luminescent material includes a host material and a guest material, wherein the host material includes a biphenyl derivative as shown in formula (1):

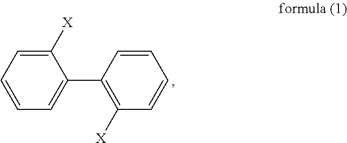

wherein X represents one of the groups shown in formula (2) to formula (5):

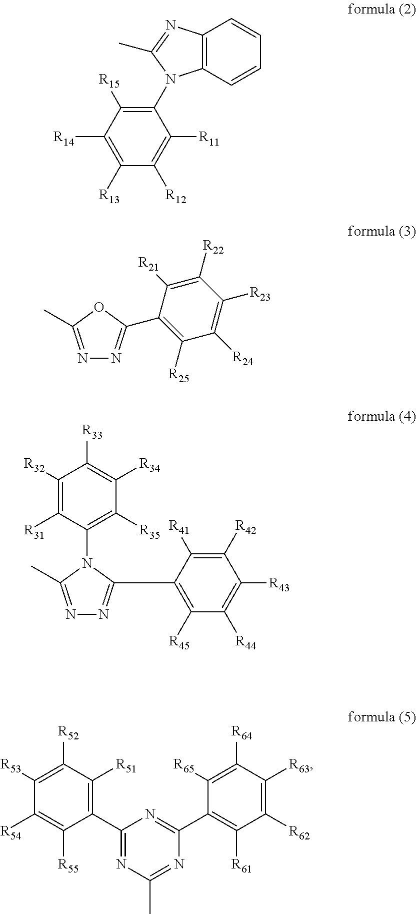

and R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group. However, the disclosure is not limited to the above. For example, X may also represent other suitable electron-accepting groups.

In this embodiment, the host material is one of the compounds shown in formula (6) to formula (11):

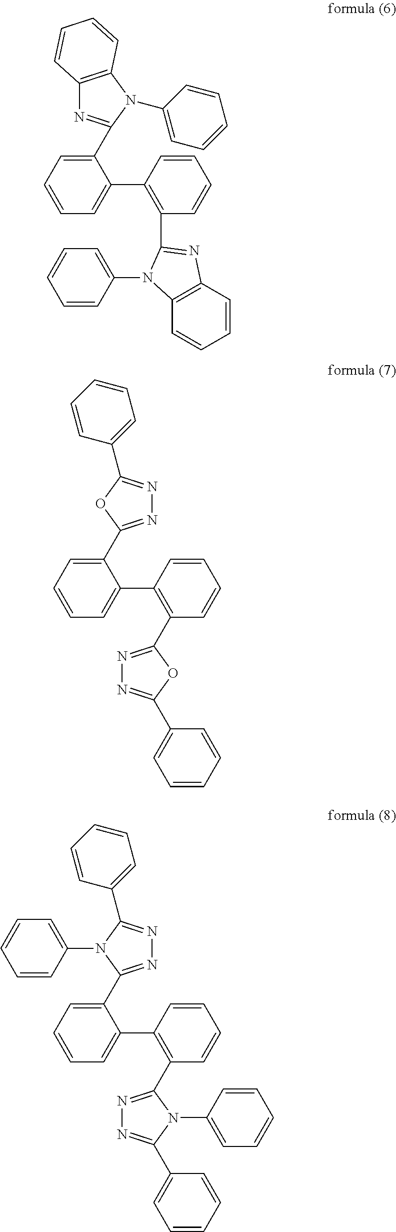 [SMILES: c1ccc(-n2c(-c3ccccc3-c3ccccc3-c3nc4ccccc4n3-c3ccccc3)nc3ccccc32)cc1 ; c1ccc(-c2nnc(-c3ccccc3-c3ccccc3-c3nnc(-c4ccccc4)o3)o2)cc1 ; c1ccc(-c2nnc(-c3ccccc3-c3ccccc3-c3nnc(-c4ccccc4)n3-c3ccccc3)n2-c2ccccc2)cc1]

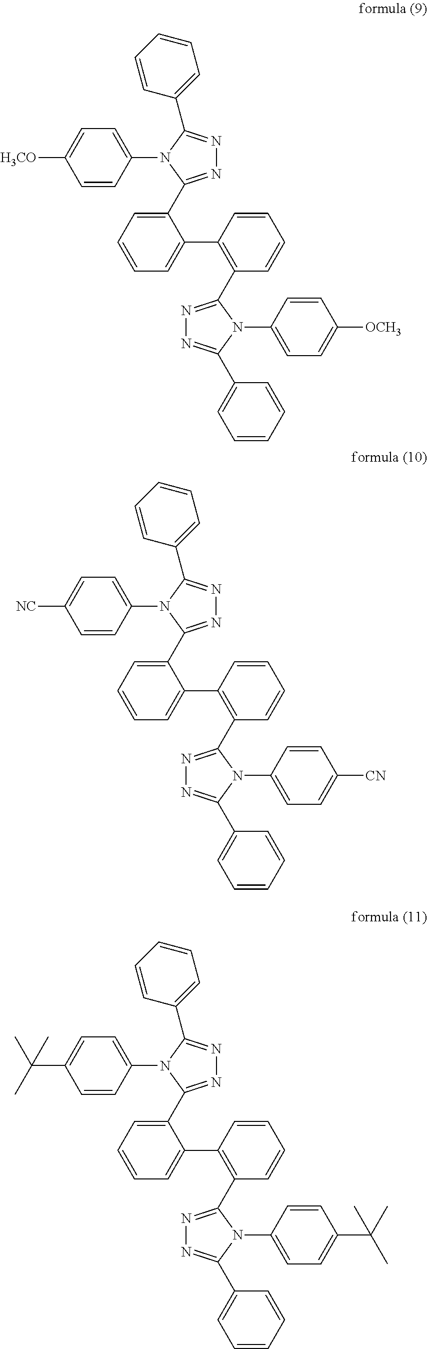 [SMILES: COc1ccc(-n2c(-c3ccccc3)nnc2-c2ccccc2-c2ccccc2-c2nnc(-c3ccccc3)n2-c2ccc(OC)cc2)cc1 ; N#Cc1ccc(-n2c(-c3ccccc3)nnc2-c2ccccc2-c2ccccc2-c2nnc(-c3ccccc3)n2-c2ccc(C#N)cc2)cc1 ; CC(C)(C)c1ccc(-n2c(-c3ccccc3)nnc2-c2ccccc2-c2ccccc2-c2nnc(-c3ccccc3)n2-c2ccc(C(C)(C)C)cc2)cc1]

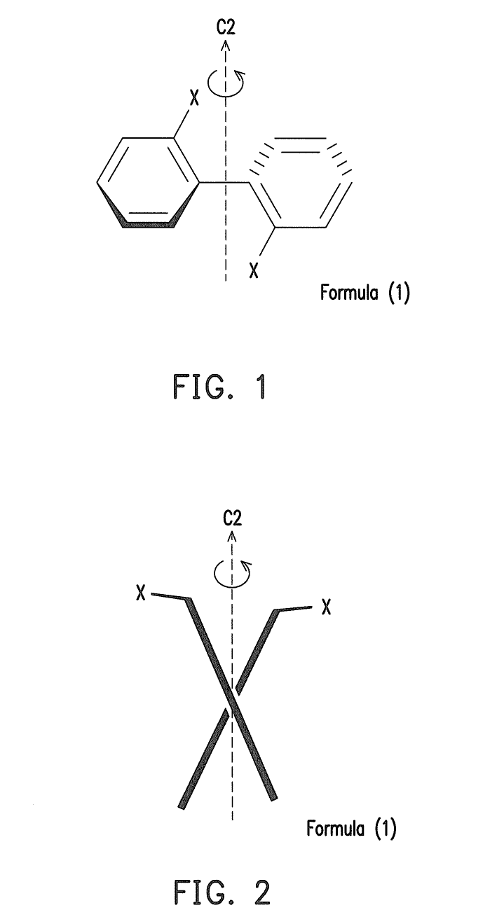

It is also noted that, for improving the luminescence efficiency of an organic luminescent layer, a triplet energy level of the host material is higher than or equal to a triplet energy level of the guest material, so as to prevent reverse energy transfer, which reduces the luminescence efficiency of a luminescent device. In this embodiment, with reference to FIG. 1 and FIG. 2, FIG. 1 and FIG. 2 are schematic top view and side view that illustrate a molecular structure of the biphenyl derivative according to one embodiment of the disclosure. The compounds shown in formula (1) and formula (6) to formula (11) are all formed with biphenyl as the center and connect an electron-accepting group at a 2,2′ position (i.e. X position in formula (1)) of the biphenyl. More specifically, as illustrated in FIG. 1 and FIG. 2, the molecular structure of the biphenyl derivative of this embodiment has a C2 rotation axis. In other words, the biphenyl derivative of this embodiment generates a steric hindrance by introducing the electron-accepting group, such that the molecule has a scissor-shaped structure, and this low-plane structure reduces a conjugation length of the biphenyl derivative of this embodiment. Thus, the host material that includes the biphenyl derivative of formula (1) of this embodiment has high triplet energy level for preventing the aforementioned reverse energy transfer, thereby improving the luminescence efficiency of the organic electroluminescent device.

Further, the guest material of this embodiment may be any material that is suitable to be used in the organic luminescent layer of the organic electroluminescent device, which may be one of the components shown in formula (12) (Ir(2-phq)₃), formula (13) (Ir(ppy)₃), and formula (14) (FIrpic). Nevertheless, the disclosure is not limited to the foregoing.

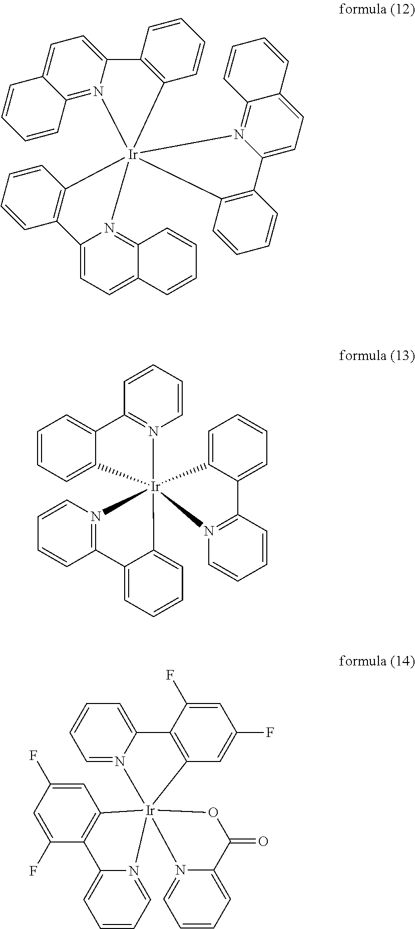

It is also noted that, according to the disclosure, the material including the biphenyl derivative shown in formula (1) can not only be used as the host material of the organic luminescent layer but also be used to form films/layers in an organic luminescent unit, such as hole injection layer, hole transport layer, electron blocking layer, electron injection layer, or electron transport layer, specifically.

### [Organic Electroluminescent Device]

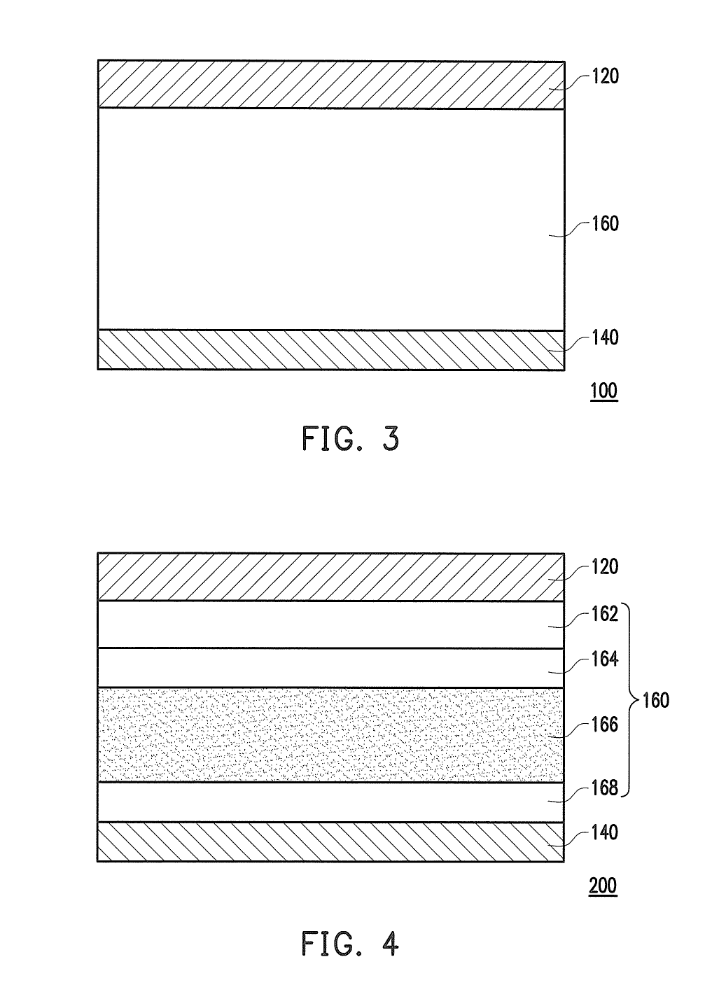

The disclosure further provides an organic electroluminescent device. FIG. 3 is a schematic cross-sectional view of an organic electroluminescent device 100 according to one embodiment of the disclosure. Referring to FIG. 3, the organic electroluminescent device 100 includes a first electrode layer 120, a second electrode layer 140, and an organic luminescent unit 160. According to this embodiment, the first electrode layer 120 is a transparent electrode material, which is ITO or other suitable metal oxide materials, for example. The second electrode layer 140 is formed of a metal, a transparent electrical conductor, or other suitable electrically conductive materials. However, the disclosure is not limited to the above. In other embodiments, the first electrode layer 120 may be a metal, a transparent electrical conductor, or other suitable electrically conductive materials while the second electrode layer 140 may be a transparent electrode material, for example. More specifically, at least one of the first electrode layer 120 and the second electrode layer 140 of this embodiment is a transparent electrode material. Accordingly, light from the organic luminescent unit 160 is emitted through the transparent electrode and causes the organic electroluminescent device 100 to emit light.

Moreover, FIG. 4 is a schematic cross-sectional view of an organic electroluminescent device 200 according to another embodiment of the disclosure. With reference to FIG. 4, the organic electroluminescent device 200 is similar to the organic electroluminescent device 100. Therefore, the same or similar components are denoted using the same or similar reference numerals and details thereof are not repeated hereinafter. The organic luminescent unit 160 of the organic electroluminescent device 200 includes a hole transport layer 162, an electron blocking layer 164, an organic luminescent layer 166, and an electron transport layer 168.

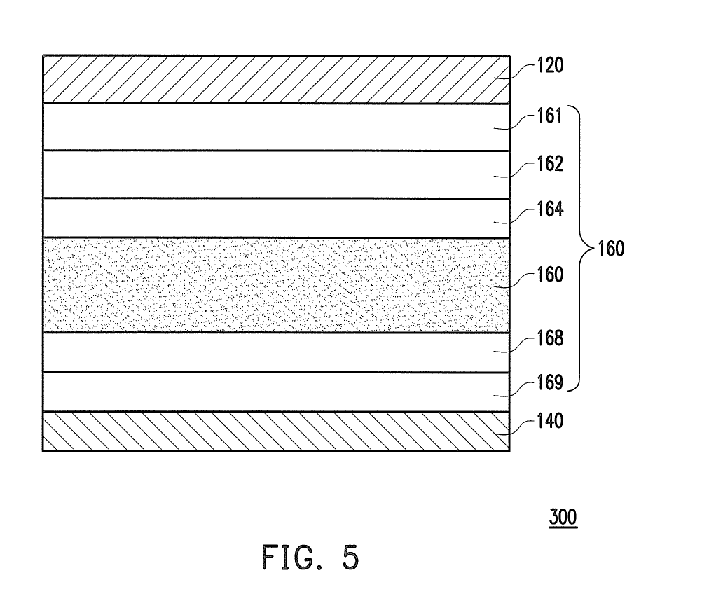

FIG. 5 is a schematic cross-sectional view of an organic electroluminescent device according to yet another embodiment of the disclosure. With reference to FIG. 5, the organic electroluminescent device 300 is similar to the organic electroluminescent device 100. Therefore, the same or similar components are denoted using the same or similar reference numerals and details thereof are not repeated hereinafter. The organic luminescent unit 160 of the organic electroluminescent device 300 includes a hole injection layer 161, a hole transport layer 162, an electron blocking layer 164, an organic luminescent layer 166, an electron transport layer 168, and a hole injection layer 169.

The organic luminescent layer 166 is disposed between the electron blocking layer 164 and the electron transport layer 168. In this embodiment, the thickness of the organic luminescent layer 166 is in a range of 5 nm to 60 nm. The organic luminescent layer 166 includes a host material and a guest material. In this embodiment, the host material includes the biphenyl derivative of formula (1).

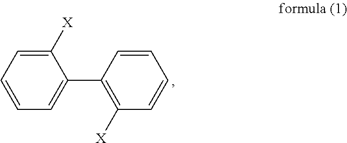

In formula (1), X represents one of the groups shown in formula (2) to formula (5).

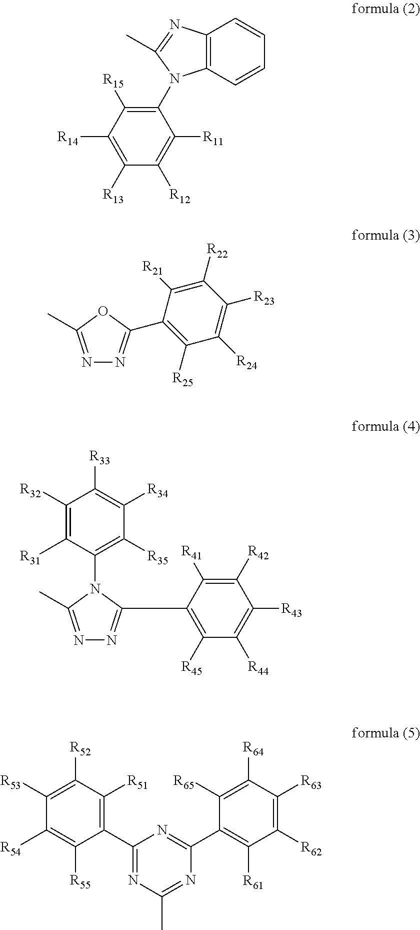

In formula (1) to formula (5), R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group. However, the disclosure is not limited to the above. For example, X may also represent other suitable electron-accepting groups.

According to one embodiment of the disclosure, the host material of the organic luminescent layer 166 includes one of the biphenyl derivatives shown in formula (6) to formula (11).

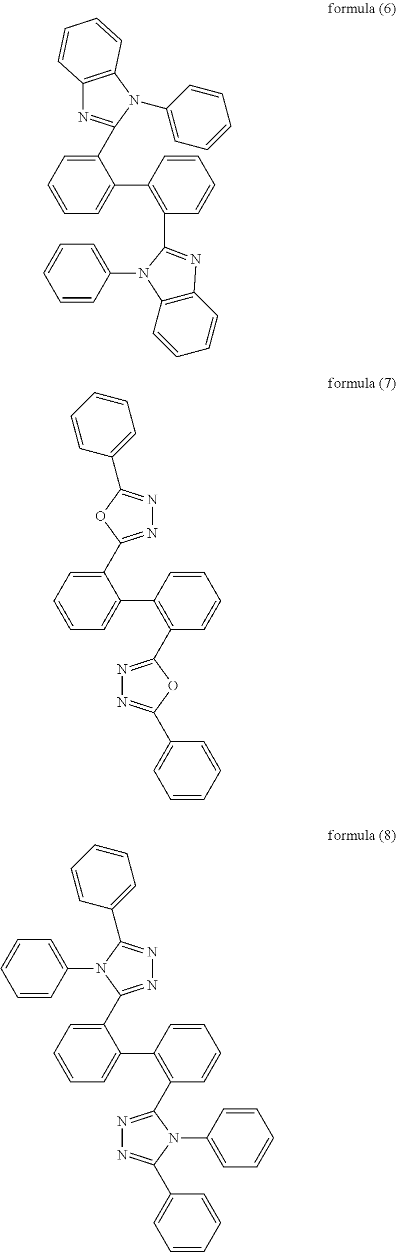 [SMILES: c1ccc(-n2c(-c3ccccc3-c3ccccc3-c3nc4ccccc4n3-c3ccccc3)nc3ccccc32)cc1 ; c1ccc(-c2nnc(-c3ccccc3-c3ccccc3-c3nnc(-c4ccccc4)o3)o2)cc1 ; c1ccc(-c2nnc(-c3ccccc3-c3ccccc3-c3nnc(-c4ccccc4)n3-c3ccccc3)n2-c2ccccc2)cc1]

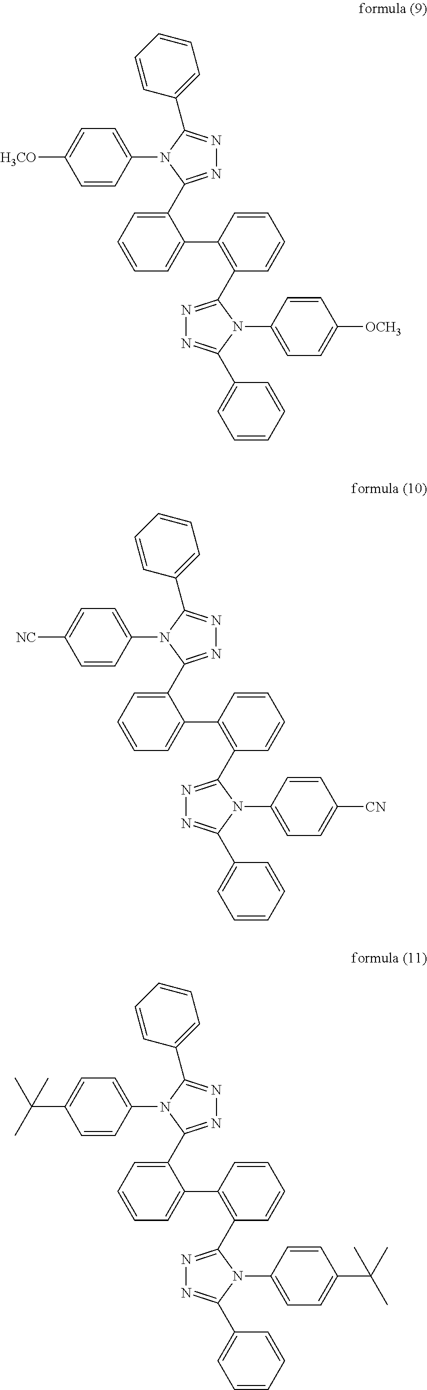 [SMILES: COc1ccc(-n2c(-c3ccccc3)nnc2-c2ccccc2-c2ccccc2-c2nnc(-c3ccccc3)n2-c2ccc(OC)cc2)cc1 ; N#Cc1ccc(-n2c(-c3ccccc3)nnc2-c2ccccc2-c2ccccc2-c2nnc(-c3ccccc3)n2-c2ccc(C#N)cc2)cc1 ; CC(C)(C)c1ccc(-n2c(-c3ccccc3)nnc2-c2ccccc2-c2ccccc2-c2nnc(-c3ccccc3)n2-c2ccc(C(C)(C)C)cc2)cc1]

According to this embodiment, a proportion of the host material that includes any one of the biphenyl derivatives of formula (1) to formula (15) to the organic luminescent layer 166 is 60% by volume to 99.5% by volume, for example.

In this embodiment, the guest material is one of the compounds shown in formula (12) to formula (14); however, the disclosure is not limited thereto.

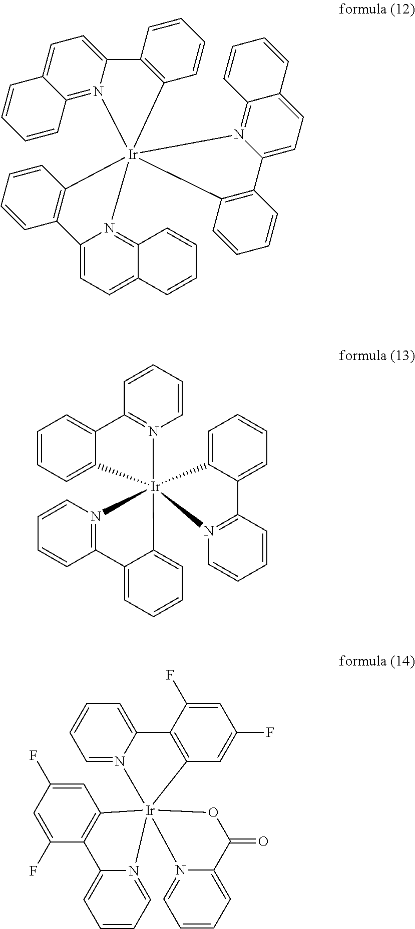

According to this embodiment, a proportion of the guest material to the organic luminescent layer 166 is 0.5% by volume to 40% by volume, for example.

Referring to FIG. 5, the hole transport layer 162 of the organic electroluminescent device 300 is disposed between the hole injection layer 161 and the electron blocking layer 164. The hole transport layer 162 is formed of a known material, such as N,N′-diphenyl-N,N′-bis(1-naphthyl)(1,1′-biphenyl)-4,4′diamine (NPB) or N,N′-diphenyl-N,N′-bis(3-methylphenyl)-1,1′-biphenyl-4,4′-diamine (TPD), for example. In this embodiment, the thickness of the hole transport layer 162 is in a range of 0 nm to 100 nm, for example. The hole transport layer 162 is adapted for increasing a speed of injecting a hole from the first electrode layer 120 into the organic luminescent layer 166 and reducing a driving voltage of the organic electroluminescent device 300.

Referring to FIG. 5, the electron blocking layer 164 is disposed between the hole transport layer 162 and the organic luminescent layer 166. A material of the electron blocking layer 164 is 1,3-bis(carbazol-9-yl)benzene (mCP) or a material having low electron affinity, for example. In this embodiment, the thickness of the electron blocking layer 164 is in a range of 0 nm to 30 nm, for example. The electron blocking layer 164 further increases the speed of transporting the hole from the hole transport layer 162 to the organic luminescent layer 166.

Referring to FIG. 5, the electron transport layer 168 is disposed between the organic luminescent layer 166 and the electron injection layer 169. A material of the electron transport layer 168 is a metal complex, such as AlQ and BeBq₂, or a heterocyclic compound, such as PBD, TAZ, and TPBI, for example. In this embodiment, the thickness of the electron transport layer 168 is in a range of 0 nm to 100 nm, for example. The electron transport layer 168 facilitates transporting electrons from the second electrode layer 140 to the organic luminescent layer 166, so as to raise the speed of electron transport.

Several synthesis examples are given below to explain the procedures of preparing the biphenyl derivatives of formula (6) to formula (11) according to the disclosure.

### SYNTHESIS EXAMPLE 1

### Synthesis of the Compound of Formula (6)

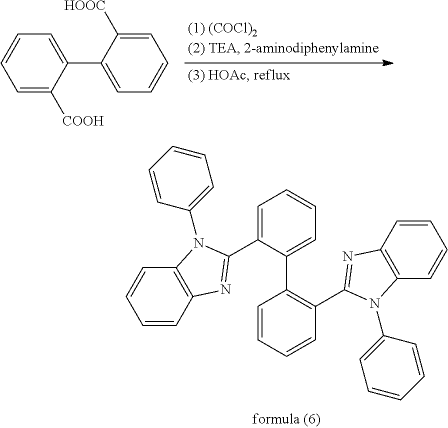 [SMILES: O=C(O)c1ccccc1-c1ccccc1C(=O)O ; c1ccc(-n2c(-c3ccccc3-c3ccccc3-c3nc4ccccc4n3-c3ccccc3)nc3ccccc32)cc1]

2,2′-biphenyl dicarboxylic acid (5.00 g, 20 mmol) and 40 ml of toluene were prepared and disposed in a 100 ml double neck bottle connected with a sodium hydroxide solution. Oxalyl chloride (7.5 ml) was added and two drops of dimethylformamide were added for catalysis. After six hours of reflux, the solvent was removed by vacuum distillation to obtain a black product. This product was dissolved in 60 ml of dichloromethane, which was slowly dropped into 60 ml of dichloromethane with triethylamine (13.75 ml) and o-amino diphenylamine (7.73 g, 42 mmol) dissolved therein for 24 hours of reflux. After reaction, extraction was performed several times using saturated brine to collect an organic layer and remove water with magnesium for filtration. The solvent was removed by rotary evaporation to obtain a black product. Acetic acid (150 ml) was added to perform 12 to 16 hours of reflux. After reaction, the solvent was removed by vacuum distillation to obtain a solid. The solid was washed with a mixture solution of ethyl acetate/ether and then filtered to obtain a gray product. The above product passed through a short column with use of an eluent of dichloromethane/ethyl acetate (3/1) to obtain a white product. At last, the above product was purified by recrystallization using a methylene chloride/ethyl acetate solution, so as to obtain the compound of formula (6) (5.8 g, yield: 54%).

### SYNTHESIS EXAMPLE 2

### Synthesis of the Compound of Formula (7)

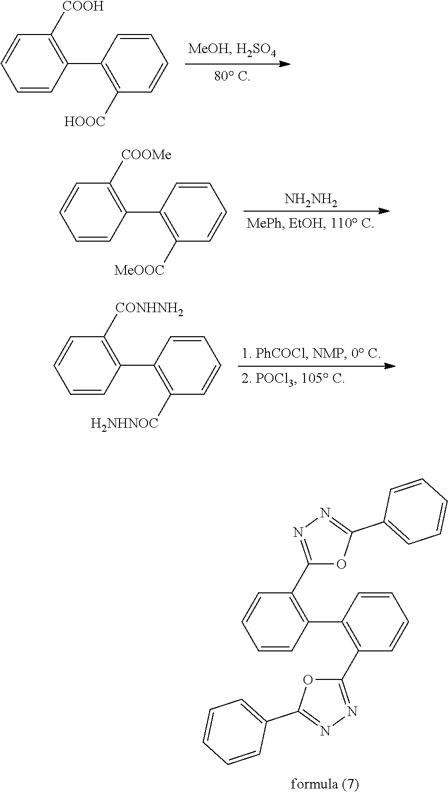

2,2′-biphenyl dicarboxylic acid (11.0 g, 45.4 mmol), methanol (50 ml), and magnetic stirrers were prepared and disposed in a 250 ml double neck bottle, and a condenser was equipped. After stirring at the room temperature for five minutes, concentrated sulfuric acid (5 ml) was added slowly and the mixture was heated to 80° C. for performing a reflux reaction for 24 hours. After the reaction was completed, a portion of the methanol was removed by a vacuum concentrator to produce a solid precipitate. Then, the white solid was collected by suction filtration, and a small amount of methanol was used to wash the solid. After removing the solvent using a vacuum system, the compound 2 (10.6 g, yield: 86%) was obtained.

Next, the compound 2 (10.0 g, 36.9 mmol), anhydrous ethanol (40 ml), toluene (40 ml), and magnetic stirrers were disposed in a 250 ml double neck bottle. 100% hydrazine hydrate (35 ml, 482.6 mmol) was injected under a nitrogen system. The mixture was heated to 110° C. for a reflux reaction for 24 hours. After the reaction was completed, the ethanol, toluene, and unreacted hydrazine hydrate were removed by atmospheric distillation. Following that, 95% ethanol was poured into the residual solid and stirred, and toluene and a small amount of ether were used in sequence to wash the solid. At last, after removing the solvent using a vacuum system, the compound 3 (8.3 g, yield: 82%) was obtained.

Then, the compound 3 (15.0 g, 55.5 mmol) and magnetic stirrers were disposed in a 500 ml double neck bottle. Dry N-methyl-2-pyrrolidone (150 ml) was injected under a nitrogen system and maintained in an ice bath for 20 minutes. Thereafter, benzoyl chloride (12.9 ml, 110.9 mmol) was injected and maintained in an ice bath for 20 minutes, and then stirred at the room temperature for 24 hours. After the reaction was completed, the above solution was slowly dropped into water to produce a white solid precipitate. Then, the white solid was collected by suction filtration to be washed with hot ethanol several times. At last, after vacuum drying, a solid of 19.7 g was obtained. The solid (0.2 g, 0.4 mmol) and magnetic stirrers were disposed in a 10 ml double neck bottle. Next, phosphorous oxychloride (4 ml) was added and a condenser was equipped, and the other end was connected to a weak base solution for heating to 105° C. and reflux for 24 hours. After the reaction was completed, the above solution was slowly poured into iced water to produce a brown solid precipitate. Then, the brown solid was collected by suction filtration to be washed with a sodium bicarbonate solution. At last, the residual water was removed using a vacuum system, and through silica gel column chromatography separation and purification, the compound of formula (7) (0.2 g, yield: 82%) was obtained.

### SYNTHESIS EXAMPLES 3 to 6

### Synthesis of the Compounds of Formula (8) to Formula (11)

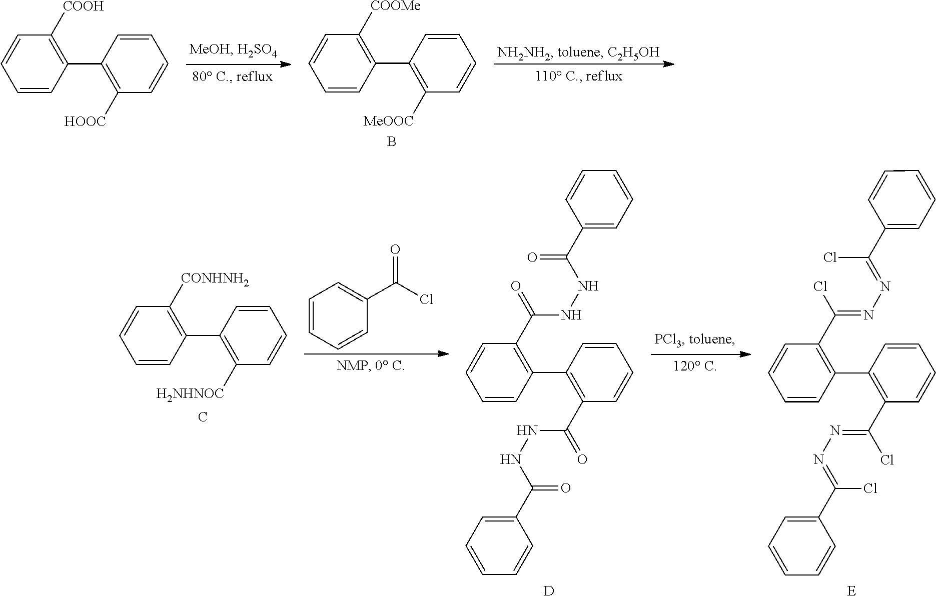

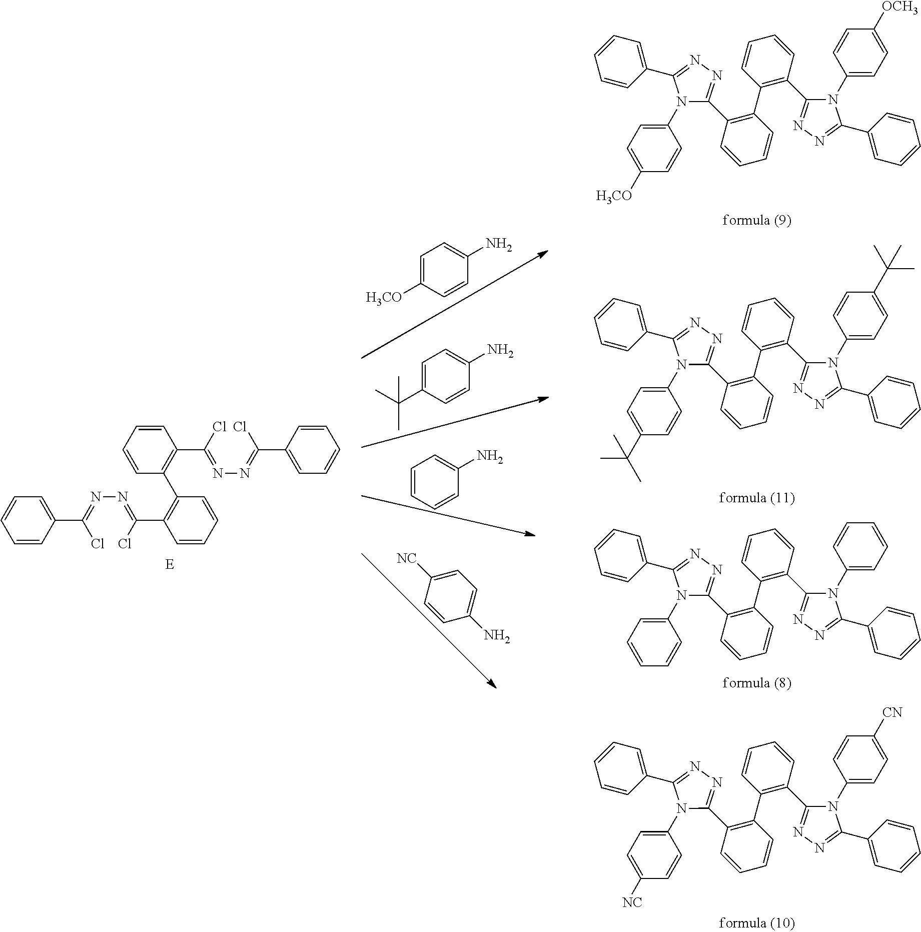

2,2′-biphenyl dicarboxylic acid was prepared to serve as an initiator, methanol was added as a solvent, and concentrated sulfuric acid of 98% of a catalytic amount was added as a dehydrating agent. The mixture was heated to 80° C. for an esterification reaction. After 24 hours of reaction, the mixture was returned to the room temperature. A portion of methanol was removed by using a rotary evaporator to form a white solid precipitate. Then, the white solid was collected by suction filtration, and a small amount of methanol was used to wash the solid. After removing the solvent using a vacuum system, a white solid compound B was obtained and the yield was 80%.

Thereafter, the compound B was obtained, and anhydrous ethanol, toluene, and 100% hydrazine hydrate were injected under a nitrogen system for performing a nucleophilic substitution reaction, heating to 110° C., and a reflux reaction for 24 hours. After the reaction was completed, the mixture was returned to the room temperature, and the ethanol, toluene, and unreacted hydrazine hydrate were removed by atmospheric distillation. Following that, 95% ethanol was poured into the residual solid and stirred. During the stirring, a white solid precipitate was formed. Then, 95% ethanol and toluene were used as a washing solution, and the white solid was collected by suction filtration. At last, after washing the solid with a small amount of ether and removing the solvent using a vacuum system, a white solid compound C was obtained and the yield was 83%.

Next, the compound C was dissolved in dry N-methyl pyrrolidone (NMP). An ice bath was performed first, and benzoyl chloride was slowly added under 0° C. for performing a nucleophilic substitution reaction. After stirring for about 20 minutes, the ice bath was removed. After 24 hours of reaction at the room temperature, the NMP solution was slowly dropped into water that was rapidly stirred by a stirrer and then left for reprecipitation. After the solid precipitated, the solid was collected by suction filtration to be washed with hot ethanol several times. Then, washing was performed with a small amount of ether. Finally, a white solid compound D was obtained and the yield was 89%.

Further, the compound D was dissolved in toluene, and phosphorus pentachloride was added under a nitrogen system. The above was heated to 120° C. for a substitution reaction for 3 hours. After the reaction was cooled, extraction was performed with toluene and water. The extraction process needs to remove all acid from the organic layer, so as to prevent influence on the subsequent recrystallization. After several extraction processes, the organic layer was taken out. After drying with use of magnesium, the liquid was collected by gravity filtration. Next, toluene was removed by a rotary evaporator to obtain a yellow solid. At last, recrystallization was performed with methylene chloride and ethanol to obtain a light yellow solid compound E and the yield was 71%.

In the Synthesis Examples 3 to 6, the compound E and an aniline derivative were dissolved in o-xylene for a cyclization reaction. The steps are explained in detail below. A triazole cyclization reaction was performed in a nitrogen system. A sand bath was heated to 160° C. for reaction for 24 hours. After the reaction was completed, the same method was used to purify and obtain the compounds of formula (8) to formula (11). More specifically, after removing the solvent by vacuum distillation, ethyl acetate was used as a washing agent and the solid was collected by suction filtration. Next, dichloromethane and ethanol were used for recrystallization. After crystallization, ethanol was used as a washing agent, and the white solid was collected by suction filtration. The compounds of formula (8), formula (9), and formula (11) were white solids, and the yields were 67%, 69%, and 72% respectively. It should be noted that, in the Synthesis Example 5, the compound of formula (10) had poor solubility and thus could not be purified by recrystallization. Therefore, after the above reaction was completed, ethyl acetate was used as the washing agent, and the white solid was collected by suction filtration. Then, after vigorously stirring with 95% ethanol and collecting the white solid by suction filtration, the white solid compound of formula (10) was obtained at a yield of 81%.

### [Method of Evaluation of the Host Material]

A method of evaluating the host material includes respectively measuring the triplet energy level (E_{T}), glass transition temperature (T_{g}), thermal decomposition temperature (T_{d}), highest occupied molecular orbital energy level (HOMO), and lowest unoccupied molecular orbital energy level (LUMO) of the compounds of the above synthesis examples. Further, the known host material mCP was used as a comparative example. The glass transition temperature (T_{g}) was measured by a differential scanning calorimeter (DSC), and a thermogravimetric analyzer (TGA) was used to measure a temperature of the material when 5% by volume was lost as the thermal decomposition temperature. The results are shown in Table 1 below.

TABLE 1

| | E_{T} | T_{g} | T_{d} | HOMO | LUMO |
|---|---|---|---|---|---|
| | (eV) | (° C.) | (° C.) | (eV) | (eV) |
| Synthesis | 2.88 | — | 350 | 6.03 | 2.22 |
| Example | | | | | |
| 1: formula (6) | | | | | |
| Synthesis | 2.72 | 44.6 | 313 | 6.22 | 2.38 |
| Example | | | | | |
| 2: formula (7) | | | | | |
| Synthesis | 3.09 | 211.2 | 417 | 6.12 | 2.05 |
| Example | | | | | |
| 3: formula (8) | | | | | |
| Synthesis | 3.06 | 186.5 | 407 | 6.14 | 2.06 |
| Example | | | | | |
| 4: formula (9) | | | | | |
| Synthesis | 3.09 | 190.5 | 472 | 6.53 | 2.48 |
| Example | | | | | |
| 5: formula (10) | | | | | |
| Synthesis | 3.10 | 280.0 | 414 | 6.09 | 2.09 |
| Example | | | | | |
| 6: formula (11) | | | | | |
| Comparative | 2.90 | 55 | — | 5.9 | 2.4 |
| Example | | | | | |

It should be noted that FIrpic was used as the guest material, for example. Referring to Table 1, although the triplet energy level (2.9 eV) of the Comparative Example is slightly higher than the triplet energy level (2.7 eV) of FIrpic, the glass transition temperature of the Comparative Example is only 55° C. . Thus, the thermal stability is low. In comparison with the glass transition temperature of the Comparative Example (55° C.), the compounds of formula (8) to formula (11) of the Synthesis Examples have high glass transition temperatures (180° C. or above). The reason is that the compounds of formula (8) to formula (11) are formed with biphenyl as the center and a large group is introduced at the 2,2′-position of the biphenyl, causing the molecules to present a non-coplanar structure. Therefore, for the compounds of formula (8) to formula (11), molecules do not easily stack to form crystallization and accordingly have better thermal stability.

Moreover, it is known from Table 1 that the thermal decomposition temperatures of the compounds of formula (6) to formula (11) were all 300° C. or above. The reason is that their structures all include multiple benzene rings, which are rigid structures and do not thermally decompose due to high temperature during the heating process. Based on the above, the biphenyl derivatives of formula (6) to formula (11) have favorable thermal stability and high triplet energy level, and therefore are suitable to serve as the host material in the organic luminescent layer of an organic light emitting diode.

Examples are provided below to explain the organic electroluminescent devices that use the biphenyl derivatives of the above Synthesis Examples as the host material and to show the luminescence efficiency of the devices.

### EXAMPLE 1

### Organic Electroluminescent Device Using the Biphenyl Derivative of Formula (6) as the Host Material

An evaporation method was used to manufacture the organic electroluminescent device. A material of the first electrode layer was ITO. A material of the second electrode layer was aluminum. NPB was used to form the hole transport layer. mCP was used as the electron blocking layer to facilitate hole injection and prevent electron from entering the hole transport layer from the luminescent layer. In the luminescent layer, the biphenyl derivative of formula (6), obtained in Synthesis Example 1, was used as the host material, and FIrpic of different doping ratios (the compound of formula (14)) was used as the guest material. A material of the electron transport layer was TAZ. The driving voltage (V), maximum current efficiency (cd/A), maximum power efficiency (lm/W), and maximum external quantum efficiency (EQE)(%) of the organic electroluminescent device manufactured in Example 1 were respectively evaluated under a current density of 20 mA/cm². The Comparative Example was an organic electroluminescent device, in which mCP was used as the host material and FIrpic of 15% by volume was used as the guest material. Evaluation results are shown in Table 2.

TABLE 2

| | FIrpic | Driving | Current | Power | |
|---|---|---|---|---|---|
| | ratio | voltage | efficiency | efficiency | EQE |
| Device | (Vol %) | (V) | (cd/A) | (lm/W) | (%) |
| 6-1 | 0% | 7.56 | 1.03 (at 5.0 V) | 0.68 (at 4.5 V) | 1.48 |
| 6-2 | 9% | 9.15 | 38.09 (at 4.5 V) | 29.19 (at 4.0 V) | 16.10 |
| 6-3 | 12% | 9.09 | 39.84 (at 5.0 V) | 29.79 (at 4.0 V) | 16.90 |
| 6-4 | 15% | 8.77 | 39.53 (at 5.0 V) | 29.02 (at 4.0 V) | 16.38 |
| 6-5 | 18% | 8.88 | 39.35 (at 5.0 V) | 29.26 (at 4.0 V) | 16.26 |
| Com- | 15% | 8.72 | 28.90 (at 6.0 V) | 16.72 (at 5.0 V) | 10.66 |
| par- | | | | | |
| ative | | | | | |
| Exam- | | | | | |
| ple | | | | | |

It is known from the above Table 2 that the organic electroluminescent device 6-3 had the greatest current efficiency, the greatest power efficiency, and the highest EQE. Thus, for the organic electroluminescent device using the biphenyl derivative of formula (6) as the host material, the optimal FIrpic doping ratio was 12%. It is worth mentioning that, under the condition of the same FIrpic doping ratio (15% by volume), the current efficiency of the organic electroluminescent device, which used the biphenyl derivative of formula (6) as the host material, was about 1.4 times the current efficiency of the organic electroluminescent device, which used mCP as the host material (Comparative Example). In addition, even if the FIrpic doping ratio was slightly adjusted, the organic electroluminescent device, which used the biphenyl derivative of formula (6) as the host material, still had greater current efficiency than the Comparative Example.

### EXAMPLE 2 TO EXAMPLE 4

### Organic Electroluminescent Devices Using the Biphenyl Derivatives of Formula (8), Formula (9), and Formula (11) as the Host Material

Organic electroluminescent devices 8, 9, and 11 were respectively manufactured by using the biphenyl derivatives of formula (8), formula (9), and formula (11), obtained in the Synthesis Examples 3, 4, and 6, as the host material and using 12% FIrpic as the guest material. The compound of formula (10) obtained in Synthesis Example 5 was used as the hole blocking layer; a material of the hole transport layer was 4,7-diphenyl-1,10-phenanthroline (BPhen); and the other layers were the same as those of Example 1. The driving voltage (V), maximum current efficiency under 1000 nits (cd/A), maximum power efficiency under 1000 nits (lm/W), and maximum brightness (cd/m²) of the organic electroluminescent devices 8, 9, and 11 were respectively evaluated under a current density of 10 mA/cm². Evaluation results are shown in Table 3.

TABLE 3

| | Driving | | Current | Power |
|---|---|---|---|---|
| | voltage | Brightness | efficiency | efficiency |
| Device | (V) | (cd/m²) | (cd/A) | (lm/W) |
| 8 | 10.7 | 2312 (at 10.5 V) | 37.9 | 23.8 |
| 9 | 9.2 | 2224 (at 11.5 V) | 24.0 | 16.2 |
| 11 | 8.3 | 2057 (at 11.4 V) | 22.2 | 15.3 |

It is known from the above Table 3 that the organic electroluminescent devices 8, 9, and 11 had low driving voltages. Moreover, in comparison with the Comparative Example of Example 1, the organic electroluminescent devices 8, 9, and 11 had higher current efficiency.

To conclude the above, the biphenyl derivative of the disclosure is formed with biphenyl as the center, and the steric hindrance is generated by introducing the electron-accepting group at the 2,2′ position of the biphenyl, such that the biphenyl derivative has a reduced conjugation length and higher triplet energy level. Therefore, when the biphenyl derivative of the disclosure is used as the host material, reverse energy transfer is prevented to improve the luminescence efficiency of the organic electroluminescent device. Further, the organic luminescent material including the biphenyl derivative of the disclosure has a high molecular weight and thus has higher glass transition temperature. In other words, the organic luminescent material of the disclosure has favorable thermal stability and is suitable for the organic electroluminescent device.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A biphenyl derivative, comprising a structure shown in formula (1): 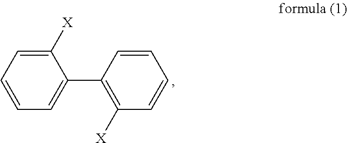
wherein X represents one of groups shown in formula (2) to formula (5): 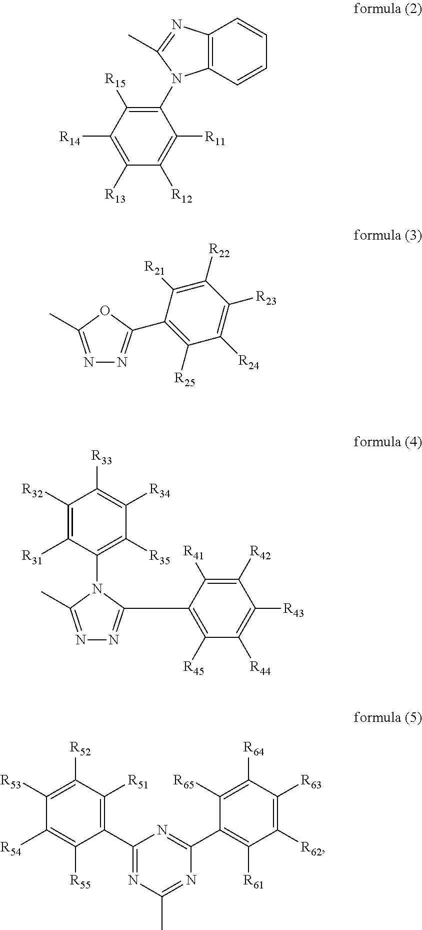
and
R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group.

2. The biphenyl derivative according to claim 1, comprising one of compounds shown in formula (6) to formula (11):

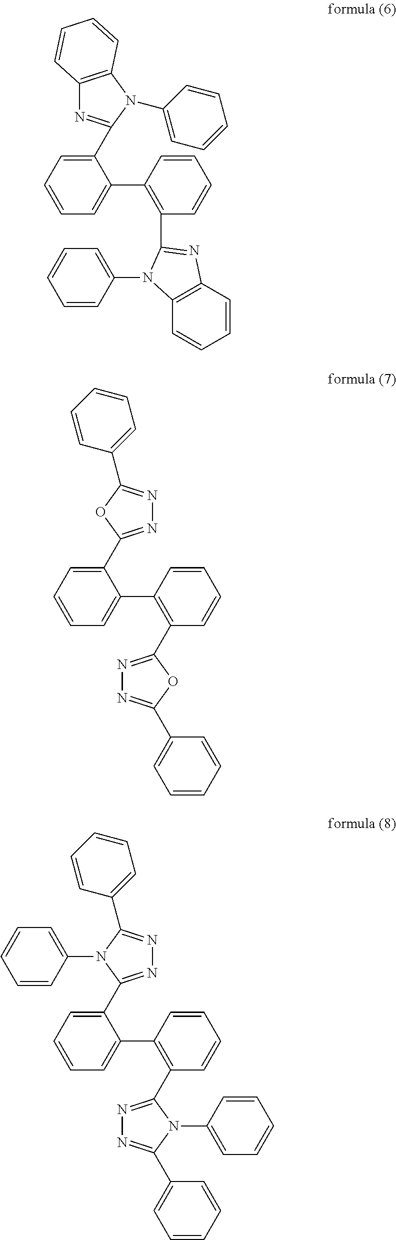 [SMILES: c1ccc(-n2c(-c3ccccc3-c3ccccc3-c3nc4ccccc4n3-c3ccccc3)nc3ccccc32)cc1 ; c1ccc(-c2nnc(-c3ccccc3-c3ccccc3-c3nnc(-c4ccccc4)o3)o2)cc1 ; c1ccc(-c2nnc(-c3ccccc3-c3ccccc3-c3nnc(-c4ccccc4)n3-c3ccccc3)n2-c2ccccc2)cc1]

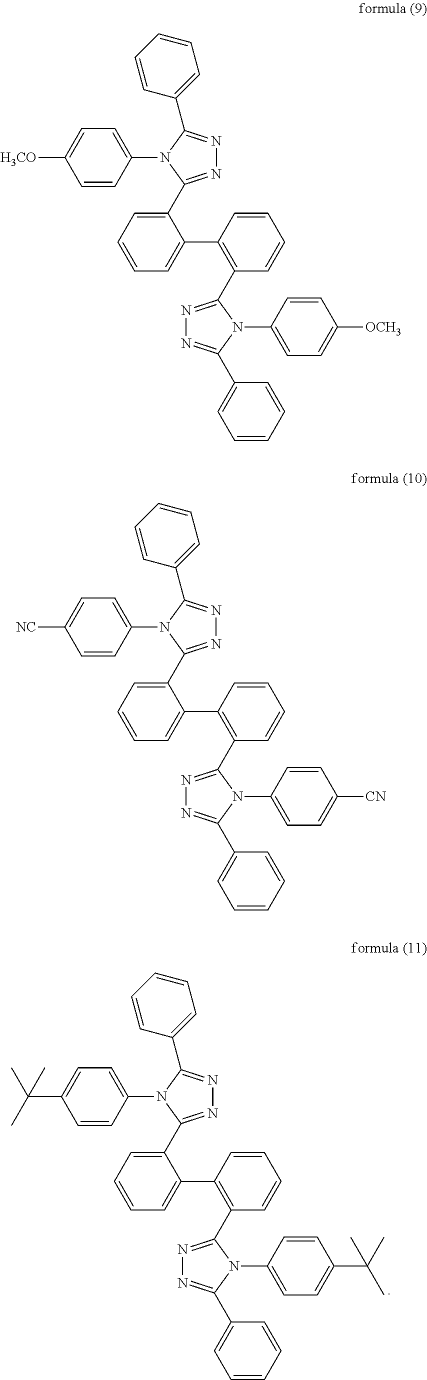 [SMILES: COc1ccc(-n2c(-c3ccccc3)nnc2-c2ccccc2-c2ccccc2-c2nnc(-c3ccccc3)n2-c2ccc(OC)cc2)cc1 ; N#Cc1ccc(-n2c(-c3ccccc3)nnc2-c2ccccc2-c2ccccc2-c2nnc(-c3ccccc3)n2-c2ccc(C#N)cc2)cc1 ; CC(C)(C)c1ccc(-n2c(-c3ccccc3)nnc2-c2ccccc2-c2ccccc2-c2nnc(-c3ccccc3)n2-c2ccc(C(C)(C)C)cc2)cc1]

3. An organic electroluminescent device, comprising:
a first electrode layer;
a second electrode layer; and
an organic luminescent unit disposed between the first electrode layer and the second electrode layer and comprising a biphenyl derivative shown in formula (1): 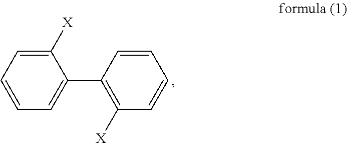
wherein X represents one of groups shown in formula (2) to formula (5): 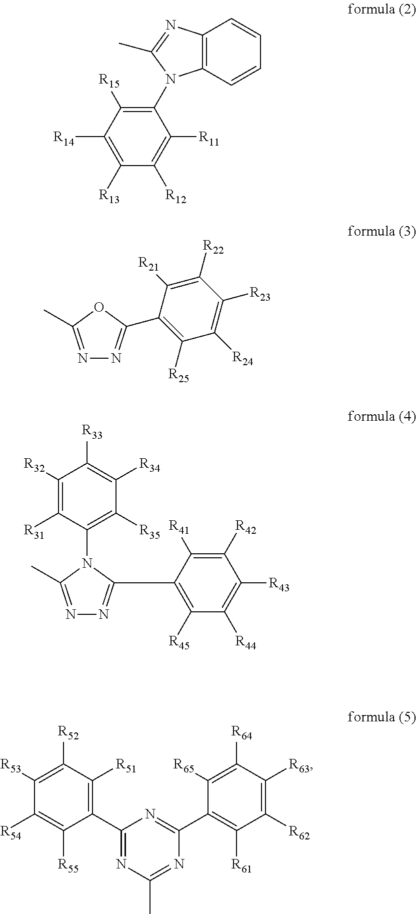
and
R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group.

4. The organic electroluminescent device according to claim 3, wherein the biphenyl derivative comprises one of compounds shown in formula (6) to formula (11):

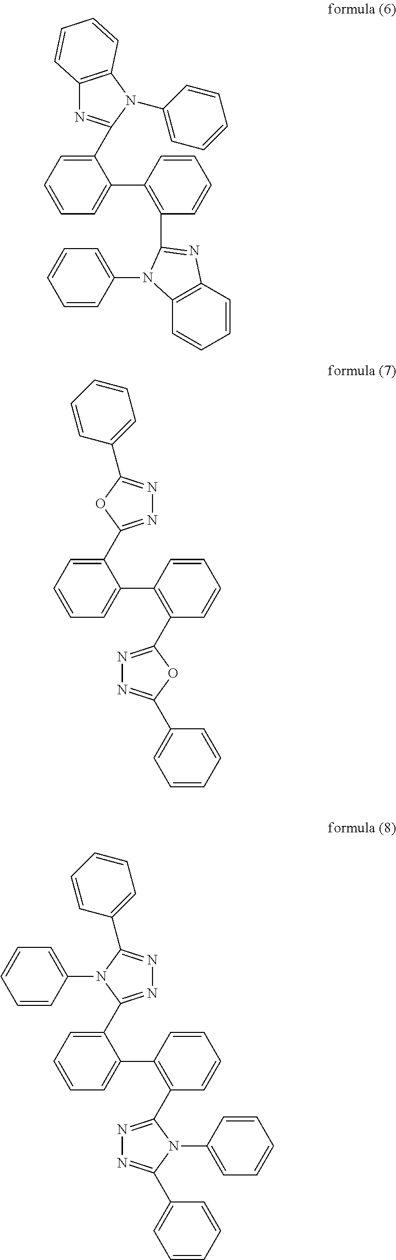 [SMILES: c1ccc(-n2c(-c3ccccc3-c3ccccc3-c3nc4ccccc4n3-c3ccccc3)nc3ccccc32)cc1 ; c1ccc(-c2nnc(-c3ccccc3-c3ccccc3-c3nnc(-c4ccccc4)o3)o2)cc1 ; c1ccc(-c2nnc(-c3ccccc3-c3ccccc3-c3nnc(-c4ccccc4)n3-c3ccccc3)n2-c2ccccc2)cc1]

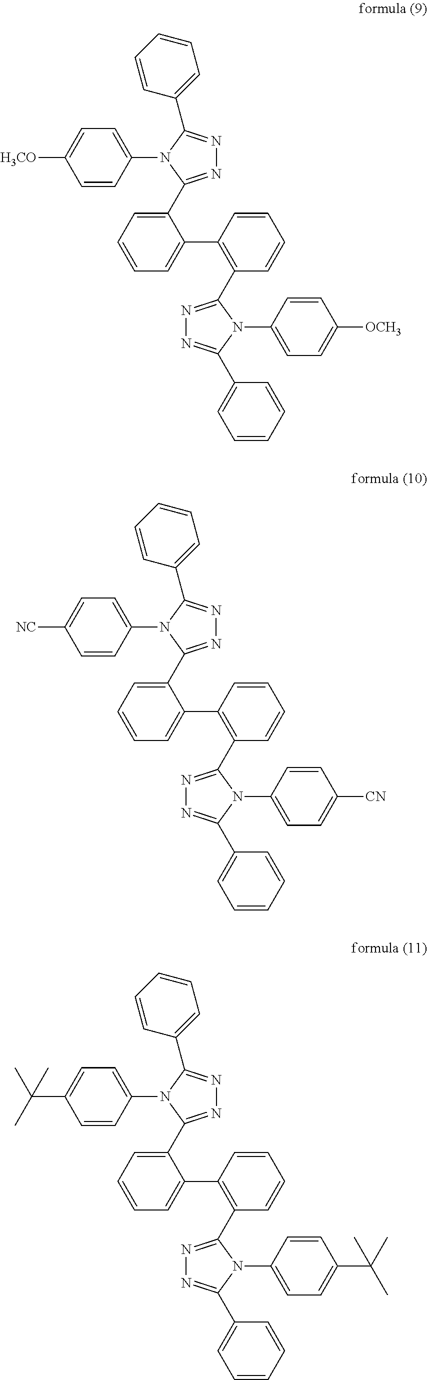 [SMILES: COc1ccc(-n2c(-c3ccccc3)nnc2-c2ccccc2-c2ccccc2-c2nnc(-c3ccccc3)n2-c2ccc(OC)cc2)cc1 ; N#Cc1ccc(-n2c(-c3ccccc3)nnc2-c2ccccc2-c2ccccc2-c2nnc(-c3ccccc3)n2-c2ccc(C#N)cc2)cc1 ; CC(C)(C)c1ccc(-n2c(-c3ccccc3)nnc2-c2ccccc2-c2ccccc2-c2nnc(-c3ccccc3)n2-c2ccc(C(C)(C)C)cc2)cc1]

5. The organic electroluminescent device according to claim 3, wherein the organic luminescent unit comprises an organic luminescent layer.

6. The organic electroluminescent device according to claim 3, wherein the organic luminescent unit further comprises a hole injection layer, a hole transport layer, an electron blocking layer, an electron injection layer, and an electron transport layer, or a combination of thereof.

7. An organic electroluminescent device, comprising:
a first electrode layer;
a second electrode layer; and
an organic luminescent unit disposed between the first electrode layer and the second electrode layer and comprising an organic luminescent layer, wherein the organic luminescent layer comprises a host material and a guest material, and the host material comprises a biphenyl derivative shown in formula (1): 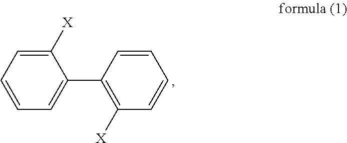
wherein X represents one of groups shown in formula (2) to formula (5): 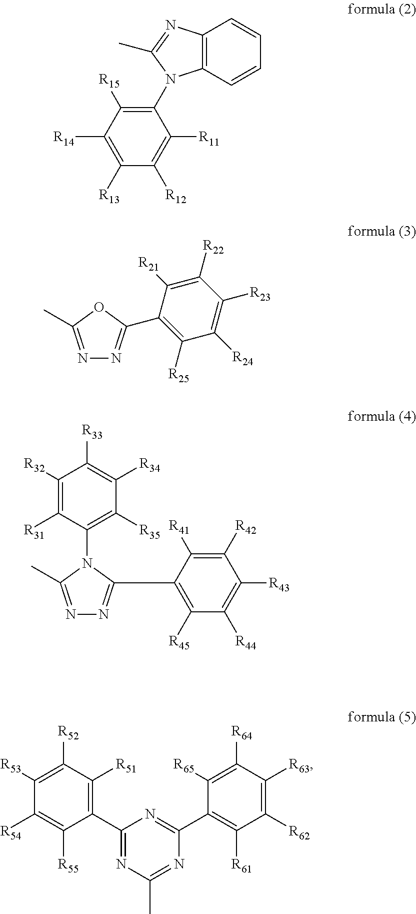
and
R₁₁, R₁₂, R₁₃, R₁₄, R₁₅, R₂₁, R₂₂, R₂₃, R₂₄, R₂₅, R₃₁, R₃₂, R₃₃, R₃₄, R₃₅, R₄₁, R₄₂, R₄₃, R₄₄, R₄₅, R₅₁, R₅₂, R₅₃, R₅₄, R₅₅, R₆₁, R₆₂, R₆₃, R₆₄, and R₆₅ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group.

8. The organic electroluminescent device according to claim 7, wherein the biphenyl derivative of the host material of the organic luminescent layer comprises one of compounds shown in formula (6) to formula (11):

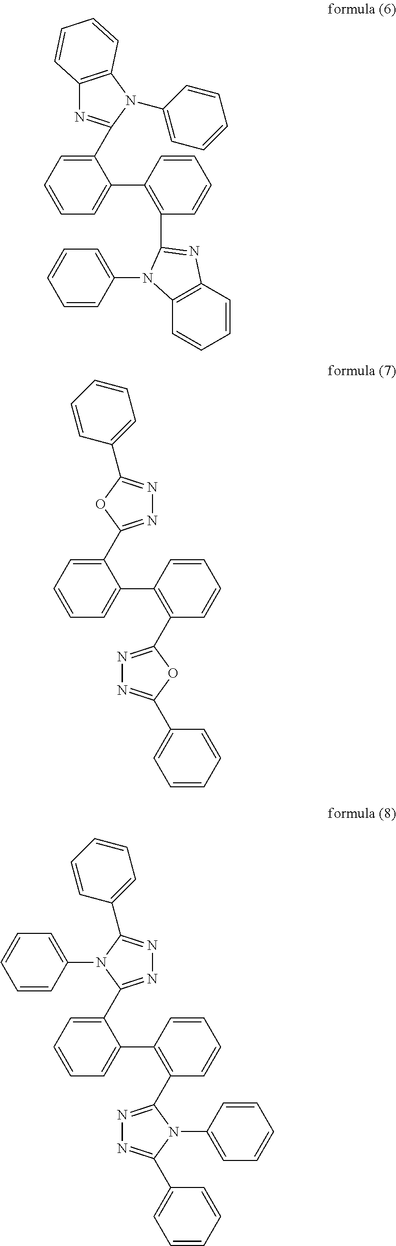 [SMILES: c1ccc(-n2c(-c3ccccc3-c3ccccc3-c3nc4ccccc4n3-c3ccccc3)nc3ccccc32)cc1 ; c1ccc(-c2nnc(-c3ccccc3-c3ccccc3-c3nnc(-c4ccccc4)o3)o2)cc1 ; c1ccc(-c2nnc(-c3ccccc3-c3ccccc3-c3nnc(-c4ccccc4)n3-c3ccccc3)n2-c2ccccc2)cc1]

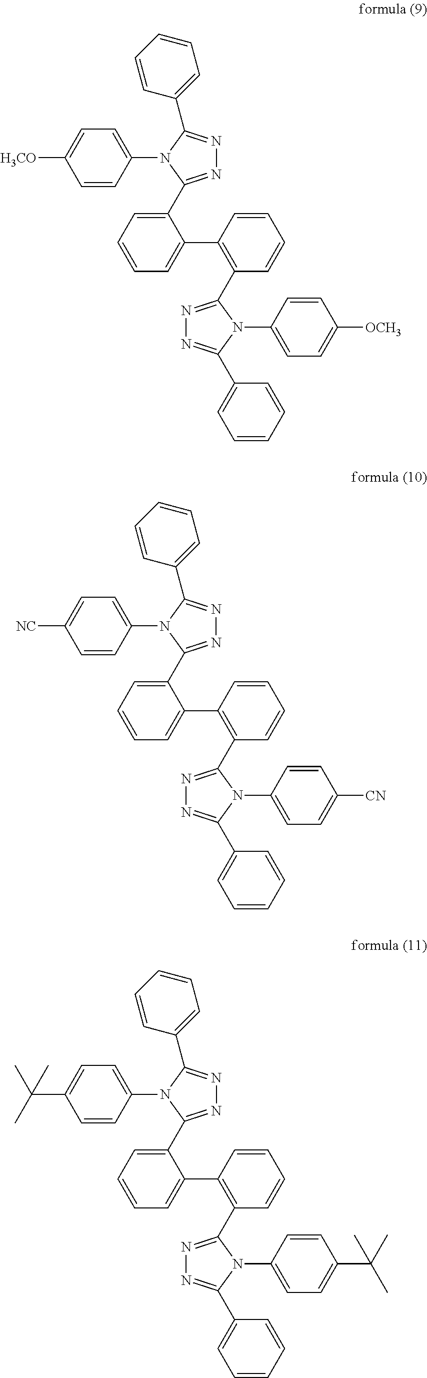 [SMILES: COc1ccc(-n2c(-c3ccccc3)nnc2-c2ccccc2-c2ccccc2-c2nnc(-c3ccccc3)n2-c2ccc(OC)cc2)cc1 ; N#Cc1ccc(-n2c(-c3ccccc3)nnc2-c2ccccc2-c2ccccc2-c2nnc(-c3ccccc3)n2-c2ccc(C#N)cc2)cc1 ; CC(C)(C)c1ccc(-n2c(-c3ccccc3)nnc2-c2ccccc2-c2ccccc2-c2nnc(-c3ccccc3)n2-c2ccc(C(C)(C)C)cc2)cc1]

9. The organic electroluminescent device according to claim 7, wherein a proportion of the host material to the organic luminescent layer is 60% by volume to 95% by volume.

10. The organic electroluminescent device according to claim 7, wherein the guest material comprises one of compounds shown in formula (12) to formula (14):

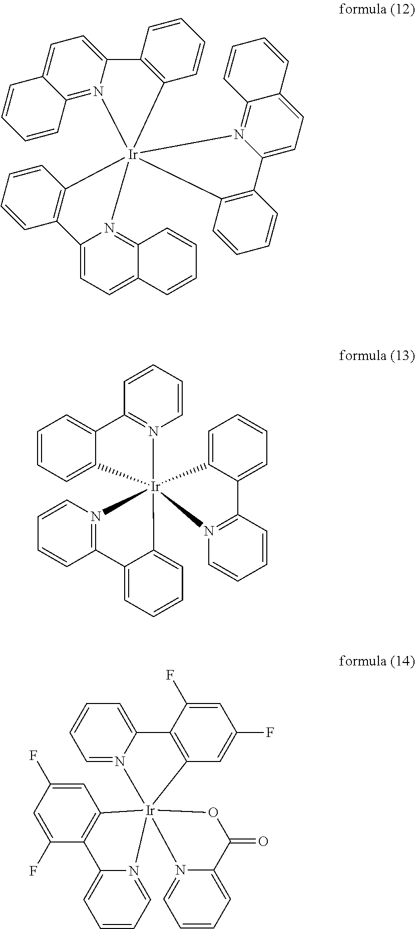

11. The organic electroluminescent device according to claim 7, wherein a proportion of the guest material to the organic luminescent layer is 5% by volume to 40% by volume.

12. The organic electroluminescent device according to claim 7, wherein at least one of the first electrode layer and the second electrode layer is a transparent electrode material.
